SEQUENCE LISTING (25 sequences)

SEQ ID NO: 1 - protein, 21 aa, synthetic construct
  source 1..21: note = CD8-derived signal peptide
MALPVTALLLPLALLLHAARP

SEQ ID NO: 2 - protein, 10 aa, synthetic construct
  source 1..10: note = Myc tag
EQKLISEEDL

SEQ ID NO: 3 - protein, 5 aa, synthetic construct
  source 1..5: note = Amino acid sequence of VH CDR1, KRAS G12V-Anti
GSYIH

SEQ ID NO: 4 - protein, 17 aa, synthetic construct
  source 1..17: note = Amino acid sequence of VH-CDR2, KRAS G12V-Anti
YIDPEYGYSRYADSVKG

SEQ ID NO: 5 - protein, 9 aa, synthetic construct
  source 1..9: note = Amino acid sequence of VH-CDR3, KRAS G12V-Anti
DSDSDAMDV

SEQ ID NO: 6 - protein, 119 aa, synthetic construct
  source 1..119: note = Full-length amino acid sequence of heavy chain variable region, KRAS G12V-Anti
ASEVQLESGGGLVQPGGSLLRSCAASGFNINGSYIHWVRQAPGKGLEWVAYIDPEYGYSRYADSVKGRFTISKNTSADTAYLQMNSLRATAEDVYYCSRDSDSDAMDVWGQGTLVTVSS

SEQ ID NO: 7 - protein, 119 aa, synthetic construct
  source 1..119: note = Full-length amino acid sequence of heavy chain variable region, KRAS G12V-Anti
ASEVQLESGSGLVQPGGSLRLSCCASGFHNIGSYIHWVAQPAGKGLKWVRYIDPEYGYSRYADSVKGRFAISADMSKNTAYLQMNSLRAEDTAVYYYSRDSDSDAMDVWGQGTLVVVSS

SEQ ID NO: 8 - protein, 11 aa, synthetic construct
  source 1..11: note = Amino acid sequence of VL CDR1, KRAS G12V-Anti
RASQDVNVAVA

SEQ ID NO: 9 - protein, 7 aa, synthetic construct
  source 1..7: note = Amino acid sequence of VL CDR2, KRAS G12V-Anti
SSSFLYS

SEQ ID NO: 10 - protein, 9 aa, synthetic construct
  source 1..9: note = Amino acid sequence of VL CDR3, KRAS G12V-Anti
QQYYYPYPT

SEQ ID NO: 11 - protein, 109 aa, synthetic construct
  source 1..109: note = Full-length amino acid sequence of light chain variable region, KRAS G12V-Anti
DIQMTQSSSSLSASVGDRVTITCRASQDVNVAVAWYQQKPGKAPKLLIYSSSFLYSGVPSRFSGSRSGTDFTLSSLQTIPEDFATYYCQQYYYPYPTFGTGQKVEIKRT

SEQ ID NO: 12 - protein, 109 aa, synthetic construct
  source 1..109: note = Full-length amino acid sequence of light chain variable region, KRAS G12V-Anti
DIQMTQMPSSLSASVGDRVTIACRASQDVNVAVAWYQQKPGKAPKLILYSSSFLYSGVPSRFSRFGSGQDFTTTISSLQPEDFATAYCQQYYYPYPTFGAGQKVEIKRT

SEQ ID NO: 13 - protein, 12 aa, synthetic construct
  source 1..12: note = Amino acid sequence of linker, KRAS G12V-Anti
GGGSGGGGSGGG

SEQ ID NO: 14 - protein, 240 aa, synthetic construct
  source 1..240: note = Amino acid sequence of single-chain fragment variable, KRAS G12V-Anti
DIQMTQSSSSLSASVGDRVTITCRASQDVNVAVAWYQQKPGKAPKLLIYSSSFLYSGVPSRFSGSRSGTDFTLSSLQTIPEDFATYYCQQYYYPYPTFGTGQKVEIKRTGGGSGGGGSGGGASEVQLESGGGLVQPGGSLLRSCAASGFNINGSYIHWVRQAPGKGLEWVAYIDPEYGYSRYADSVKGRFTISKNTSADTAYLQMNSLRATAEDVYYCSRDSDSDAMDVWGQGTLVTVSS

SEQ ID NO: 15 - protein, 240 aa, synthetic construct
  source 1..240: note = Amino acid sequence of single-chain fragment variable, KRAS G12V-Anti
DIQMTQMPSSLSASVGDRVTIACRASQDVNVAVAWYQQKPGKAPKLILYSSSFLYSGVPSRFSRFGSGQDFTTTISSLQPEDFATAYCQQYYYPYPTFGAGQKVEIKRTGGGSGGGGSGGGASEVQLESGSGLVQPGGSLRLSCCASGFHNIGSYIHWVAQPAGKGLKWVRYIDPEYGYSRYADSVKGRFAISADMSKNTAYLQMNSLRAEDTAVYYYSRDSDSDAMDVWGQGTLVVVSS

SEQ ID NO: 16 - protein, 45 aa, synthetic construct
  source 1..45: note = CD8alpha-derived hinge domain
TTTPAPRPPTPAPTIASQPLSLRPEACRPAAGGAVHTRGLDFACD

SEQ ID NO: 17 - protein, 68 aa, synthetic construct
  source 1..68: note = Amino acid sequence of CD28-derived transmembrane domain
FWVLVVVGGVLACYSLLVTVAFIIFWVRSKRSRLLHSDYMNMTPRRPGPTRKHYQPYAPPRDFAAYRS

SEQ ID NO: 18 - protein, 24 aa, synthetic construct
  source 1..24: note = Amino acid sequence of CD8-derived transmembrane domain
IYIWAPLAGTCGVLLLSLVITLYC

SEQ ID NO: 19 - protein, 42 aa, synthetic construct
  source 1..42: note = Amino acid sequence of 4-1BB-derived intracellular signaling domain
KRGRKKLLYIFKQPFMRPVQTTQEEDGCSCRFPEEEEGGCEL

SEQ ID NO: 20 - protein, 112 aa, synthetic construct
  source 1..112: note = Amino acid sequence of CD3zeta-derived intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 21 bp, Homo sapiens
  source 1..21: mol_type = genomic DNA; note = KRAS sequence of pancreatic cancer cell line CFPAC-1 (contiguous nucleotide sequence including 3 codons before and after G12)
gttggagctgttggcgtaggc

SEQ ID NO: 22 - DNA, 21 bp, Homo sapiens
  source 1..21: mol_type = genomic DNA; note = KRAS sequence of pancreatic cancer cell line PANC-1 (contiguous nucleotide sequence including 3 codons before and after G12)
gttggagctgatggcgtaggc

SEQ ID NO: 23 - DNA, 21 bp, Homo sapiens
  source 1..21: mol_type = genomic DNA; note = KRAS sequence of pancreatic cancer cell line BxPC-3 (contiguous nucleotide sequence including 3 codons before and after G12)
gttggagctggtggcgtaggc

SEQ ID NO: 24 - DNA, 20 bp, synthetic construct
  source 1..20: mol_type = other DNA; note = Forward Primer for KRAS sequencing
ccaggcctgctgaaaatgac

SEQ ID NO: 25 - DNA, 20 bp, synthetic construct
  source 1..20: mol_type = other DNA; note = Reverse primer for KRAS sequencing
tggtccctcattgcactgta

(19) United States
(12) Patent Application Publication
(10) Pub. No.: US 2026/0108609 A1
(43) Pub. Date: Apr. 23, 2026

(54) SINGLE-CHAIN FRAGMENT VARIABLE TARGETING KRAS G12V, CHIMERIC ANTIGEN RECEPTOR, AND USE THEREOF

(71) Applicant: THE FIRST AFFILIATED HOSPITAL OF WANNAN MEDICAL COLLEGE, Wuhu, Anhui (CN)

(72) Inventors: Zhenyu XU, Wuhu, Anhui (CN); Huimin SHAO, Wuhu, Anhui (CN)

(21) Appl. No.: 19/132,969

(22) PCT Filed: May 31, 2024
(86) PCT No.: PCT/CN2024/096542

§ 371 Date: May 27, 2025

(30) Foreign Application Priority Data
Jun. 1, 2023 (CN) 202310640046.9

(51) Int. Cl. A61K 40/42; A61K 40/11; A61K 40/31; A61P 35/00; C07K 14/725; C07K 16/40

(52) U.S. Cl. CPC A61K 40/4253; A61K 40/11; A61K 40/31; A61P 35/00; C07K 14/7051; C07K 16/40; C07K 2317/569

(57) ABSTRACT

Disclosed are a single-chain fragment variable (scFv) targeting KRAS G12V, a chimeric antigen receptor (CAR), and a use thereof. Based on the KRAS G12V target, a T cell receptor (TCR) is modified. An extracellular signaling domain of the TCR for recognizing a tumor-specific antigen (TSA) is retained and linked in series to an extracellular spacer, a transmembrane domain, and a CD3ζ-derived intracellular signaling domain in the conventional CAR structure, such that the modified CAR can specifically recognize a KRAS G12V mutant polypeptide presented by HLA-A*02:01. Moreover, based on the advantages of CAR-T cell/NK cell therapy, potential new tumor treatment options are explored to lay a foundation for clinical trials.

### CROSS REFERENCE TO THE RELATED APPLICATIONS

This application is the national phase entry of International Application No. PCT/CN2024/096542, filed on May 31, 2024, which is based upon and claims priority to Chinese Patent Application No. 202310640046.9, filed on Jun. 1, 2023. The entire contents of International Application No. PCT/CN2024/096542 and Chinese Patent Application No. 202310640046.9 are incorporated herein by reference.

### SEQUENCE LISTING

The instant application contains a Sequence Listing which has been submitted in XML format via EFS-Web and is hereby incorporated by reference in its entirety. Said XML copy is named SequenceListing.xml, created on May 23, 2025, and is 29,506 bytes in size.

### TECHNICAL FIELD

The present disclosure relates to a single-chain fragment variable (scFv) targeting KRAS G12V, a chimeric antigen receptor (CAR), and a use thereof, and belongs to the field of biomedicine.

### BACKGROUND

Cell therapy has become one of the most attractive fields in recent years. The cell therapy has exhibited a promising therapeutic prospect in cancers, hematological diseases, cardiovascular diseases, diabetes, neurological diseases, etc., and has played an important role in the immunotherapy for various diseases, especially for tumors. At present, there have been more than ten cellular immunotherapies commercially available on the market. Cellular immunotherapies exhibit prominent therapeutic effects in hematological and lymphatic tumors, but still suffer from some limitations in the treatment of solid tumors. Currently, those used in clinical trials are mostly tumor-associated antigens (TAAs), such as mesothelin (MSLN), epidermal growth factor receptor (EGFR), and claudin 18.2 (CLDN18.2). However, TAAs are also expressed in some normal tissues, which poses off-target risks and safety issues to cell therapies. Therefore, the selection of highly-specific targets for solid tumors is the primary problem that needs to be solved urgently.

Tumor antigens are classified into TAAs and tumor-specific antigens (TSAs). TAAs can be expressed by both tumor cells and normal cells, but the levels of TAAs are significantly increased when normal cells undergo carcinogenesis. TAAs only exhibit changes in their levels and are not strictly tumor-specific. TSAs are a class of antigens that are unique to tumor cells or are present only in a particular type of tumor cells but not in normal cells. Neoantigens are a class of TSAs derived from non-synonymous mutations, and are very attractive targets for the immunotherapy of tumors. Unlike TAAs, TSAs/neoantigens are mutant peptides produced from genetic alterations in tumor genomes, and TSAs/neoantigens are specifically expressed in tumor cells and are absent in normal tissues. Thus, TSAs/neoantigens exhibit high tumor specificity, and can reduce the off-target toxicity. A personalized immunotherapy based on a neoantigen can invoke a robust anti-tumor immune response against tumor cells.

RAS gene is the first human oncogene ever discovered, and is also a common mutated gene in tumors. RAS gene mainly has three isoforms: HRAS (Harvey rat sarcoma viral oncogene homolog), KRAS (Kirsten rat sarcoma viral oncogene homolog), and NRAS (Neuroblastoma rat sarcoma viral oncogene homolog). KRAS is the most predominant mutant in the RAS family and is associated with 22% of human tumors. KRAS is a frequently mutated gene in the top 3 most deadly cancers in humans: lung cancer (17%), colorectal cancer (33%), and pancreatic cancer (61%). In healthy cells, KRAS functions as a switch for regulating cell growth, and plays an important role in the regulation of a variety of cell signaling events such as cell proliferation. However, when a mutation occurs in the KRAS gene, KRAS is stuck in the “on” position to lead to uncontrolled cell growth and activation of downstream signaling pathways, such that cells start to proliferate malignantly, resulting in the development and metastasis of cancer. 97% of KRAS mutations occur at codon 12 and codon 13. The most common KRAS mutations are G12C, G12D, G12V, and G13D mutations in KRAS. It has been demonstrated in clinical studies that the G12V mutation is of great research value in the immunotherapy for various cancer types, and is a desirable target for tumor immunotherapy.

CARs are synthetic receptors that can direct immune cells to specifically track, recognize, and scavenge tumor cells expressing relevant target ligands. CAR typically includes an extracellular binding domain (generally derived from a single-chain fragment variable of an antigen-binding domain of a monoclonal antibody) that can recognize a TAA, a hinge domain, a transmembrane domain, and an intracellular signaling domain of a T cell receptor (TCR) molecule, and possesses a high targeting capacity. CAR binds to a tumor antigen in an antigen-dependent manner without major histocompatibility complex (MHC) restriction, which enables CAR-engineered T cells to recognize a wider range of tumor cells compared to native T cell receptors (TCRs).

HLA-A*02 alleles are expressed in 44% of the Caucasian population and in up to 37.7% of the Chinese population. HLA-A*02:01 allele is expressed in more than 10% of the Chinese population. The affinity for the HLA-A*02:01 and the RAS G12V mutant protein is predicted with the NetMHCpan-4.0 bioinformatics algorithm. The conventional T cell receptor-engineered T (TCR-T) cells can recognize both intracellular and extracellular tumor antigens presented by MHC. The conventional TCR-T cells possess the capacity to recognize intracellular antigens having low-level mutations with ultra-high sensitivity, but are MHC-restricted. However, chimeric antigen receptor-engineered T (CAR-T) cells/natural killer (NK) cells can only recognize antigens on the surface of tumor cells.

### SUMMARY

In order to solve the above-mentioned technical problems, a first objective of the present disclosure is to provide a single-chain fragment variable targeting KRAS G12V. The single-chain fragment variable targeting KRAS G12V includes a heavy chain variable region (VH) and a light chain variable region (VL), where the heavy chain variable region includes a VH-CDR1, a VH-CDR2, and a VH-CDR3, and the light chain variable region includes a VL-CDR1, a VL-CDR2, and a VL-CDR3.

An amino acid sequence of the VH-CDR1 is a sequence set forth in SEQ ID NO: 3, an amino acid sequence of the VH-CDR2 is a sequence set forth in SEQ ID NO: 4, and an amino acid sequence of the VH-CDR3 is a sequence set forth in SEQ ID NO: 5:

SEQ ID NO: 3:
GSYIH;
SEQ ID NO: 4:
YIDPEYGYSRYADSVKG;
and
SEQ ID NO: 5:
DSDSDAMDV.

An amino acid sequence of the VL-CDR1 is a sequence set forth in SEQ ID NO: 8, an amino acid sequence of the VL-CDR2 is a sequence set forth in SEQ ID NO: 9, and an amino acid sequence of the VL-CDR3 is a sequence set forth in SEQ ID NO: 10 (QQYYYPYPT):

SEQ ID NO: 8:
RASQDVNVAVA;
SEQ ID NO: 9:
SSSFLYS;
and
SEQ ID NO: 10:
QQYYYPYPT.

Further, a full-length amino acid sequence of the heavy chain variable region is an amino acid sequence set forth in SEQ ID NO: 6:

SEQ ID NO: 6:
ASEVQLESGGGLVQPGGSLLRSCAASGFNINGSYIHWVRQAPGKGLEW
VAYIDPEYGYSRYADSVKGRFTISKNTSADTAYLQMNSLRATAEDVYY
CSRDSDSDAMDVWGQGTLVTVSS;

or
an amino acid sequence set forth in SEQ ID NO: 7:

SEQ ID NO: 7:
ASEVQLESGSGLVQPGGSLRLSCCASGFHNIGSYIHWVAQPAGKGLKWV
RYIDPEYGYSRYADSVKGRFAISADMSKNTAYLQMNSLRAEDTAVYYYS
RDSDSDAMDVWGQGTLVVVSS.

A full-length amino acid sequence of the light chain variable region is an amino acid sequence set forth in SEQ ID NO: 11:

SEQ ID NO: 11:
DIQMTQSSSSLSASVGDRVTITCRASQDVNVAVAWYQQKPGKAPKLLIY
SSSFLYSGVPSRFSGSRSGTDFTLSSLQTIPEDFATYYCQQYYYPYPTF
GTGQKVEIKRT;

or
an amino acid sequence set forth in SEQ ID NO: 12:

SEQ ID NO: 12:
DIQMTQMPSSLSASVGDRVTIACRASQDVNVAVAWYQQKPGKAPKLILY
SSSFLYSGVPSRFSRFGSGQDFTTTISSLQPEDFATAYCQQYYYPYPTF
GAGQKVEIKRT.

Further, the heavy chain variable region and the light chain variable region are linked by a linker, and the linker includes three tetrapeptide multimers, and the linker has an amino acid sequence set forth in SEQ ID NO: 13:

SEQ ID NO: 13:
GGGSGGGGSGGG.

Further, an amino acid sequence of the single-chain fragment variable is an amino acid sequence set forth in SEQ ID NO: 14 or an amino acid sequence having a homology of at least 80% with the amino acid sequence set forth in SEQ ID NO: 14:

SEQ ID NO: 14:
DIQMTQSSSSLSASVGDRVTITCRASQDVNVAVAWYQQKPGKAPKLLIY
SSSFLYSGVPSRFSGSRSGTDFTLSSLQTIPEDFATYYCQQYYYPYPTF
GTGQKVEIKRTGGGSGGGGSGGGASEVQLESGGGLVQPGGSLLRSCAAS
GFNINGSYIHWVRQAPGKGLEWVAYIDPEYGYSRYADSVKGRFTISKNT
SADTAYLQMNSLRATAEDVYYCSRDSDSDAMDVWGQGTLVTVSS;

or
an amino acid sequence set forth in SEQ ID NO: 15 or an amino acid sequence having a homology of at least 80% with the amino acid sequence set forth in SEQ ID NO: 15:

SEQ ID NO: 15:
DIQMTQMPSSLSASVGDRVTIACRASQDVNVAVAWYQQKPGKAPKLILY
SSSFLYSGVPSRFSRFGSGQDFTTTISSLQPEDFATAYCQQYYYPYPTF
GGAGQKVEIKRTGGGSGGGGSGGASEVQLESGSGLVQPGGSLRLSCCAS
GFHNIGSYIHWVAQPAGKGLKWVRYIDPEYGYSRYADSVKGRFAISADM
SKNTAYLQMNSLRAEDTAVYYYSRDSDSDAMDVWGQGTLVVVSS.

A second objective of the present disclosure is to provide a CAR, including an antigen-binding domain, a transmembrane domain, and an intracellular signaling domain, where the antigen-binding domain includes a signal peptide, a Myc tag, and the single-chain fragment variable according to claims 1 to 3 for recognizing an antigen.

Further, the signal peptide is selected from a group consisting of CD8, GM-CSF, CD4, CD28, CD137, a mutant/modified form thereof, and a combination thereof.

Further, the signal peptide is a CD8-derived signal peptide, and has an amino acid sequence set forth in SEQ ID NO: 1:

SEQ ID NO: 1:
MALPVTALLLPLALLLHAARP.

Further, the Myc tag is an epitope tag derived from a c-myc gene. The Myc tag can be fused as a fusion protein to an N-terminus or a C-terminus of the single-chain fragment variable, and has an amino acid sequence set forth in SEQ ID NO: 2:

SEQ ID NO: 2:
EQKLISEEDL.

Further, the Myc tag is fused to the N-terminus of the single-chain fragment variable.

Further, the antigen-binding domain and the transmembrane domain are linked by a hinge domain, and the hinge domain is selected from a group consisting of CD8α, a derivative of an extracellular domain of CD28, an IgG-based hinge domain including a fragment crystallizable (FC) moiety or a CH2/CH3 domain of an Fc moiety of IgG1 or IgG4, and DAP12.

Further, the hinge domain is a CD8α-derived hinge domain, and has an amino acid sequence set forth in SEQ ID NO: 16:

SEQ ID NO: 16:
TTTPAPRPPTPAPTIASQPLSLRPEACRPAAGGAVHTRGLDFACD.

Further, the transmembrane domain is one selected from a group consisting of CD3ζ, CD8, CD28, NKG2D, 2B4, and DNAM1.

Further, the transmembrane domain is a CD28-derived transmembrane domain or a CD8-derived transmembrane domain, where an amino acid sequence of the CD28-derived transmembrane domain is set forth in SEQ ID NO: 17:

SEQ ID NO: 17:
FWVLVVVGGVLACYSLLVTVAFIIFWVRSKRSRLLHSDYMNMTPRRPGP
TRKHYQPYAPPRDFAAYRS;

and/or
an amino acid sequence of the CD8-derived transmembrane domain is set forth in SEQ ID NO: 18:

SEQ ID NO: 18:
IYIWAPLAGTCGVLLLSLVITLYC.

Further, the intracellular signaling domain is one or more selected from a group consisting of CD3ζ, CD3γ, CD3δ, CD3ε, CD5, CD22, CD79a, CD79b, CD66d, CD2, CD4, CD5, a CD28 family, DAP10, DAP12, NKp44, NKG2D, a tumor necrosis factor receptor (TNFR) family, and a signaling lymphocytic activation molecule (SLAM) family of receptors.

Further, the intracellular signaling domain is a combination of a 4-1BB-derived intracellular signaling domain and a CD3ζ-derived intracellular signaling domain, where an amino acid sequence of the 4-1BB-derived intracellular signaling domain is set forth in SEQ ID NO: 19 and an amino acid sequence of the CD3ζ-derived intracellular signaling domain is set forth in SEQ ID NO: 20:

SEQ ID NO: 19:
KRGRKKLLYIFKQPFMRPVQTTQEEDGCSCRFPEEEEGGCEL;
and
SEQ ID NO: 20:
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

A third objective of the present disclosure is to provide a CAR-T cell expressing the CAR as described above.

A fourth objective of the present disclosure is to provide a use of the single-chain fragment variable targeting KRAS G12V, the CAR, and the CAR-T cell as described above in a preparation of an anti-tumor drug or an anti-tumor product.

The present disclosure achieves the following advantages. The present disclosure provides a single-chain fragment variable targeting KRAS G12V, a CAR, and a use thereof. Based on the KRAS G12V target, TCR is modified. An extracellular signaling domain of the TCR for recognizing TSA is retained and linked in series to an extracellular spacer, a transmembrane domain, and a CD3ζ-derived intracellular signaling domain in the conventional CAR structure, such that the modified CAR can specifically recognize a KRAS G12V mutant polypeptide presented by HLA-A*02:01. Moreover, based on the advantages of CAR-T cell/NK cell therapy, potential new tumor treatment options are explored to lay a foundation for clinical trials.

### BRIEF DESCRIPTION OF THE DRAWINGS

FIG. 1 illustrates structural maps of KGV1 CAR and KGV2 CAR.

FIG. 2A-FIG. 2D show lentiviral infection efficiencies of KGV1 CAR-T cell and KGV2 CAR-T cell that are detected by flow cytometry.

FIG. 3A-FIG. 3B show sorting efficiencies for positive KGV1 CAR-T cells and positive KGV2 CAR-T cells.

FIG. 4 illustrates structural schematic illustrations of a KRAS G12V plasmid, an HLA-A*02:01 plasmid, and a luciferase plasmid.

FIG. 5A-FIG. 5B show the proportion of exogenous target cells K562#4 expressing KRAS G12V mutation and HLA*02:01.

FIG. 6A-FIG. 6B show the cytotoxicity of KGV1 CAR-T cells and KGV2 CAR-T cells against exogenous target cells.

FIG. 7A-FIG. 7D show the secretion of cytokines TNF-α and IFN-γ during exogenous killing.

FIG. 8A-FIG. 8C show the phenotypic identification for endogenous target cells.

FIG. 9A-FIG. 9C show the cytotoxicity of KGV1 CAR-T cells and KGV2 CAR-T cells against endogenous target cells.

FIG. 10A-FIG. 10F show the secretion of cytokines TNF-α and IFN-γ during endogenous killing.

### DETAILED DESCRIPTION OF THE EMBODIMENTS

The present disclosure is further described below with reference to specific examples. The following examples are only provided for describing the technical solutions of the present disclosure clearly, and are not intended to limit the protection scope of the present disclosure.

The present disclosure will be further described below in conjunction with the accompanying drawings and examples.

### Example 1 Sequence Design and Vector Construction for CAR

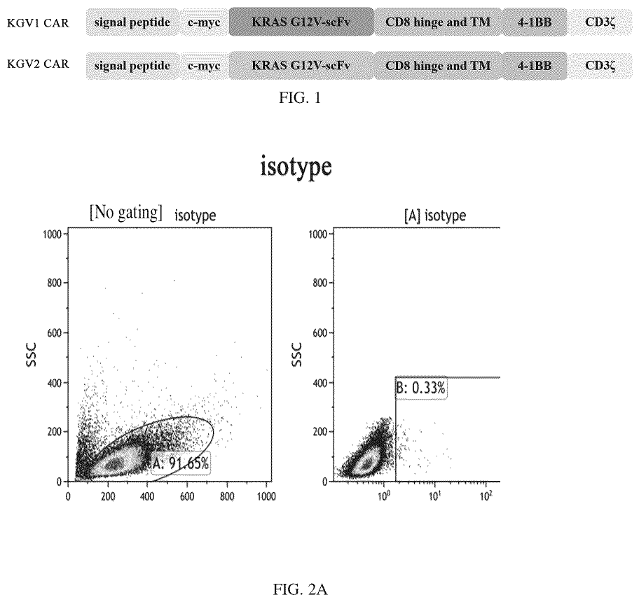

For the CAR, sequences of a signal peptide, a Myc tag, a CD8α-derived hinge domain, a CD8-derived transmembrane domain, and 4-1BB- and CD3ζ-derived intracellular signaling domains were all from Addgene as a public database, and scFv was obtained by screening a phage antibody mutant library as shown in Example 3. The signal peptide, the Myc tag, the single-chain fragment variable targeting KRAS G12V, the CD8α-derived hinge domain, the CD8-derived transmembrane domain, and the 4-1BB- and CD3ζ-derived intracellular signaling domains were ligated sequentially between EcoR I and Sal I restriction sites of a vector PCDH-EF1α to construct major expression plasmids, which were designated as KGV1 CAR and KGV2 CAR, respectively. Structural maps of the major expression plasmids are shown in FIG. 1.

The specific process for constructing the major expression plasmids could be a common method in the prior art, which would not be elaborated here.

### Example 2 Construction of a Phage Antibody Mutant Library

Peripheral blood mononuclear cells (PBMCs) were isolated from 1,000 healthy human peripheral blood samples, and mRNA was extracted and reverse-transcribed into cDNA. Based on a known antibody sequence, a target fragment was amplified by error-prone polymerase chain reaction (PCR). The resulting PCR product was subjected to 2% agarose gel electrophoresis, then a target band with a size of about 500 bp was cut out of the gel, and the product was recovered by gel extraction. The PCR product and a phagemid vector pCANTAB5E each were subjected to double-enzyme cleavage with SfiI and NotI, followed by gel extraction. T4 ligase was added in a base-pair ratio of vector:DNA of 1:3 for ligation, and the ligation was performed at 16° C. overnight in a PCR amplifier. 5 μL of a ligation product was added to 100 μL of TG1 competent cells, and the resulting mixture was pre-cooled on ice and transferred to a pre-cooled electroporation cuvette. The voltage of an electroporator was adjusted to 2.5 KV, and the electroporation was performed with electric shocks for 5 ms. After the electroporation was completed, 0.9 mL of a medium was immediately added, followed by incubation with shaking for 2 h at 37° C. 10 μL of the resulting cell suspension was taken, serially diluted, and plated on an SOBAG plate. A library capacity was calculated. Monoclones were randomly picked for sequencing to verify the diversity of a mutant antibody library.

### Example 3 Screening of the Phage Antibody Mutant Library

1.5 mL of the above mutant antibody library was inoculated into 300 mL of a medium, and cultured for about 1.5 h at 37° C. under shaking to obtain a first culture. A helper phage M13KO7 was added in a volume 5 times a volume of the first culture for superinfection, followed by incubation with shaking at 37° C. for about 1 h to obtain a second culture. The second culture was centrifuged at 4,000 rpm and 15° C. for 15 min, and the resulting supernatant (medium) was discarded. 200 mL of a medium (containing 100 μg/mL Ampicillin and 50 μg/mL Kanamycin) was added to resuspend bacterial cells and cultured at 37° C. for 2 h to obtain a third culture. The third culture was centrifuged at 10,000 rpm for 20 min to obtain a first supernatant and a first precipitate. The precipitate was discarded. 40 mL of PEG/NaCl was added to the first supernatant to precipitate phages, and immersed overnight in an ice bath, followed by centrifugation at 10,000 rpm for 20 min to obtain a second supernatant, and the second supernatant was discarded. Phages were resuspended with 0.6 mL of a medium and stored at 4° C. for later use. An enzyme linked immunosorbent assay (ELISA) microplate was coated with His Bind Resin to bind an antigen protein. The target protein as a stationary phase and the phage display library as a mobile phase were co-incubated for 2 h, and the unbound free phages were washed away. Then phages bound and adsorbed to the target molecules were eluted with an acid and then used to infect TG1 host bacteria, and the infected TG1 host bacteria were subjected to reproduction and expansion for the next round of elution. Five rounds of “adsorption-elution-expansion” were performed to enrich a specific antibody. The plating was performed, and monoclones were picked out and verified by ELISA. Finally, two scFvs targeting KRAS G12V were successfully screened out. Amino acid sequences of the two scFvs were set forth in SEQ ID NO: 14 and SEQ ID NO: 15, respectively:

SEQ ID NO: 14:
DIQMTQSSSSLSASVGDRVTITCRASQDVNVAVAWYQQKPGKAPKLLIY
SSSFLYSGVPSRFSGSRSGTDFTLSSLQTIPEDFATYYCQQYYYPYPTF
GTGQKVEIKRTGGGSGGGGSGGGASEVQLESGGGLVQPGGSLLRSCAAS
GFNINGSYIHWVRQAPGKGLEWVAYIDPEYGYSRYADSVKGRFTISKNT
SADTAYLQMNSLRATAEDVYYCSRDSDSDAMDVWGQGTLVTVSS;
and
SEQ ID NO: 15:
DIQMTQMPSSLSASVGDRVTIACRASQDVNVAVAWYQQKPGKAPKLILY
SSSFLYSGVPSRFSRFGSGQDFTTTISSLQPEDFATAYCQQYYYPYPTF
GAGQKVEIKRTGGGSGGGGSGGGASEVQLESGSGLVQPGGSLRLSCCAS
GFHNIGSYIHWVAQPAGKGLKWVRYIDPEYGYSRYADSVKGRFAISADM
SKNTAYLQMNSLRAEDTAVYYYSRDSDSDAMDVWGQGTLVVVSS.

### Example 4 Preparation of a Lentivirus

(1) 5×10⁵ HEK293F cells were inoculated into 30 mL of a medium in a shake flask and cultured for 3 d until a cell density reached about 4.0×10⁶ cells/mL to 5.0×10⁶ cells/mL, followed by lentivirus packaging. 1.5 mL of an antibiotic- and serum-free medium was added, 37.5 μg of a major expression plasmid and 37.5 μg of a packaging plasmid mixture were added successively in a ratio of 1:1, and were thoroughly mixed by gently pipetting up and down. 180 μL of a transfection reagent was added to 1.5 mL of an antibiotic- and serum-free medium, and thorough mixing was performed gently. The plasmid and the transfection reagent were thoroughly mixed and then incubated at room temperature for 10 min. The resulting transfection reagent-DNA mixture was slowly transferred to a shake flask, and the shake flask was gently shaken for thorough mixing and then placed in an incubator at 5% CO₂ and 37° C.
(2) After the transfection was performed for 48 h, the resulting supernatant culture was collected and centrifuged for 10 min at 3,000 rpm/min and 4° C. to obtain a first supernatant and a first precipitate. The first precipitate was discarded. The first supernatant was filtered through a 0.45 μm filter membrane to obtain a virus supernatant filtrate.
(3) According to a ratio of 1:4, to a viral concentrate, the virus supernatant filtrate was slowly added in a fed-batch manner, and the resulting mixture was allowed to be layered and then centrifuged for 4 h at 10,000 g/min and 4° C. to allow virus concentration to obtain a second supernatant and a second precipitate. The second supernatant was discarded. The second precipitate was retained and suspended in an antibiotic- and serum-free medium according to a ratio of virus supernatant filtrate to medium of 250:1. The resulting viral suspension was dispensed and stored at −80° C. for later use.

### Example 5 Preparation of T Cells

The T cells were derived from peripheral blood of healthy people. 15 mL of a lymphocyte separation medium warmed to room temperature was added in advance to each of eight 50 mL centrifuge tubes. 100 mL of heparinized blood was taken and thoroughly mixed with normal saline in a ratio of 1:1 to obtain diluted blood. 25 mL of the diluted blood was slowly added on the lymphocyte separation medium along a tube wall by a pipette, and then horizontally centrifuged at 650 g/min and 20° C. for 20 min. In order to ensure the separation effect, an ascending speed and a descending speed of a centrifuge should be adjusted to minimums. After the centrifugation, there were four layers in a tube, including a plasma layer, a PBMC layer, a lymphocyte separation medium layer, a granulocyte and red blood cell layer sequentially from top to bottom. The uppermost plasma layer was removed with about 1 mL left. The PBMC layer was collected in a rotary manner along a tube wall with a pipette, and transferred to a fresh 50 mL centrifuge tube. Normal saline was supplemented, and the resulting mixture was centrifuged at 470 g for 10 min. The resulting supernatant was discarded. If there was a large amount of a red substance at the bottom, a red blood cell lysis buffer could be added to lyse red blood cells. After the lysis was completed, normal saline was added, followed by centrifugation at 300 g/min for 10 min. The resulting supernatant was discarded. Normal saline was added, followed by washing once, and centrifugation at 300 g/min for 5 min. The resulting supernatant was discarded. Cells were resuspended with RPMI-1640 complete medium preheated at 37° C., and counted and calculated for the cell viability. PBMCs were isolated through the above density gradient centrifugation. Cell-stimulating factors CD3 and CD28 were then added at a concentration of 200 ng/mL to activate T cells. 24 h later, 10 ng/mL IL-7, 20 ng/mL IL-15, and 20 ng/mL IL-21 were added, and the expansion culture was continued.

### Example 6 Preparation of CAR-T Cells

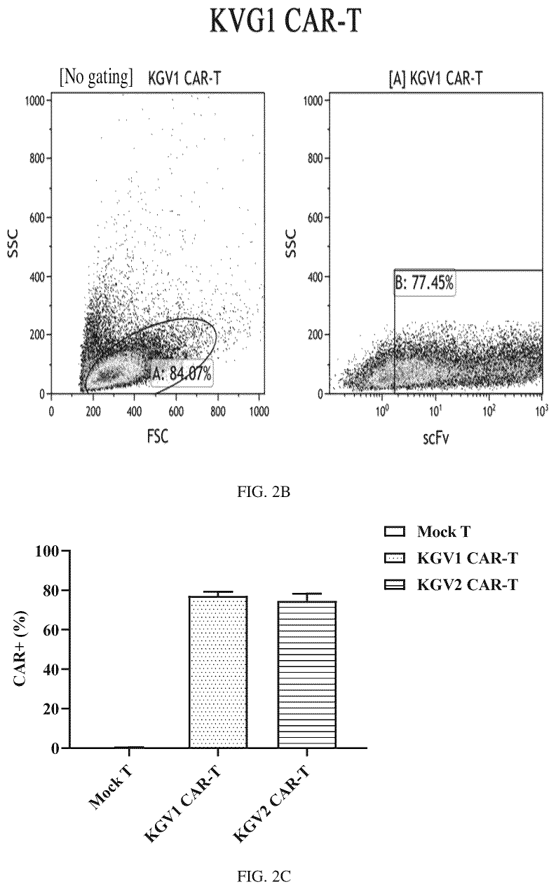

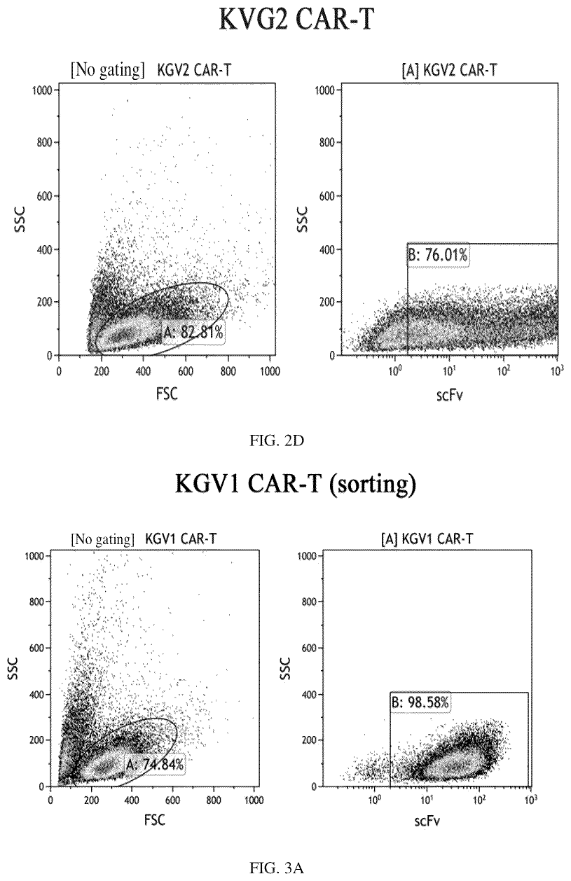

A lentiviral system was adopted to prepare the CAR-T cells. Activated T cells were added to a lentiviral concentrate at MOI=10 for viral transfection, and 5 μg/mL Polybrene (transfection enhancer) was added. 24 h later, a medium was changed, and the incubation was continued. Lentiviral infection efficiencies were detected by flow cytometry to be 77.45% and 76.01%, respectively, as shown in FIG. 2A-FIG. 2D.

### Example 7 Sorting of Positive CAR-T Cells

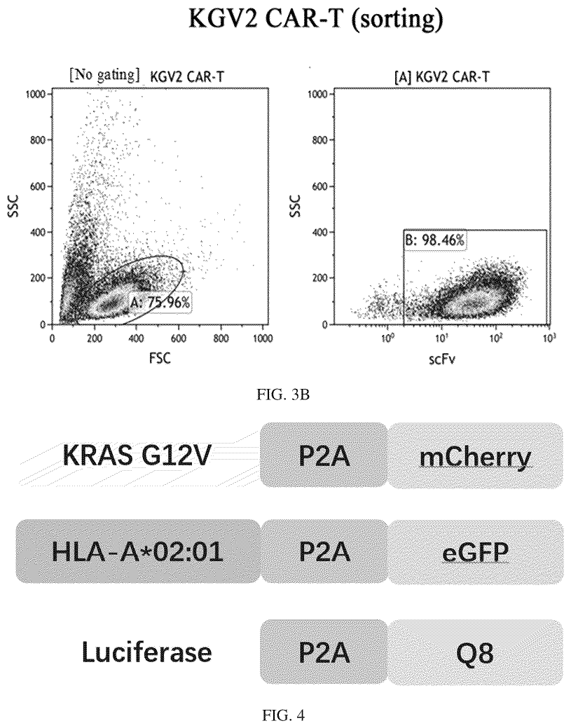

The positive CAR-T cells were sorted out with Myc-Tag (9B11) Mouse mAb (PE Conjugate) (purchased from Cell Signaling Technology) and Anti-PE MicroBeads (purchased from Miltenyi Biotec). A specific process was as follows. 5 μL of the Myc-Tag (9B11) Mouse mAb (PE Conjugate) was added to every 10 million cells, and incubation was performed at 4° C. for 30 min. Cells were washed three times with phosphate buffered saline (PBS). Then 80 μL of a sorting buffer and 20 μL of Anti-PE MicroBeads were added, and incubation was performed at 4° C. for 15 min. Cells were washed three times with PBS, and then loaded on an LS column. Cells eluted from the LS column were positive CAR-T cells. Positive rates of KGV1 CAR-T cells and KGV2 CAR-T cells produced after sorting reached 98% or more. Results are shown in FIG. 3A-FIG. 3B.

### Example 8 Construction and Verification of Target Cells Exogenously Expressing a KRAS G12V Mutation and HLA*02:01

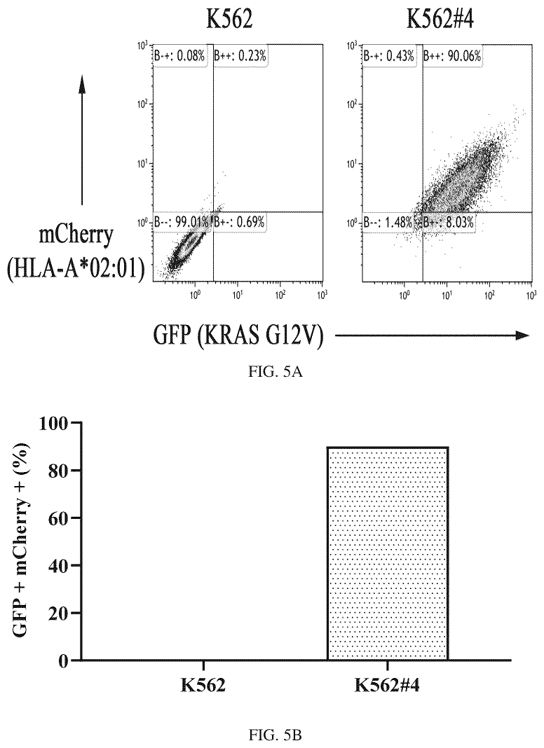

K562 cells were adopted as exogenous target cells. Because K562 cells could not express KRAS and endogenous HLA, KRAS G12V and HLA*02:01 plasmids were stably transfected into K562 cells through lentivirus infection. The expression was detected at 72 h. Transfected cells were cultured for 14 d to produce a stably-transfected cell line. Double-positive cells were sorted through fluorescence-activated cell sorting, expanded, and identified for a phenotype by flow cytometry. Results are shown in FIG. 5A-FIG. 5B. Structural schematic illustrations of a KRAS G12V plasmid, an HLA*02:01 plasmid, and a luciferase plasmid are illustrated in FIG. 4. Moreover, a luciferase-expressing transposon plasmid was electroporated into the double-positive cells for the subsequent killing function experiments, and resulting electroporated cells were named K562#4. Results were shown in Table 1.

TABLE 1
Firefly luciferase assays for K562-luc cells and K562#4-luc cells

| Cell | Cell | Reading on a | Luciferase |
|---|---|---|---|
| name | count | microplate reader | efficiency |
| K562-luc | 1*10⁵ | 15283 | 15.28% |
| K562#4-luc | 1*10⁵ | 27538 | 27.54% |
| RPMI-1640 | 0 | 8 | — |
| complete medium | | | |

### Example 9 Identification of Target Cells Endogenously Expressing a KRAS G12V Mutation and HLA*02:01

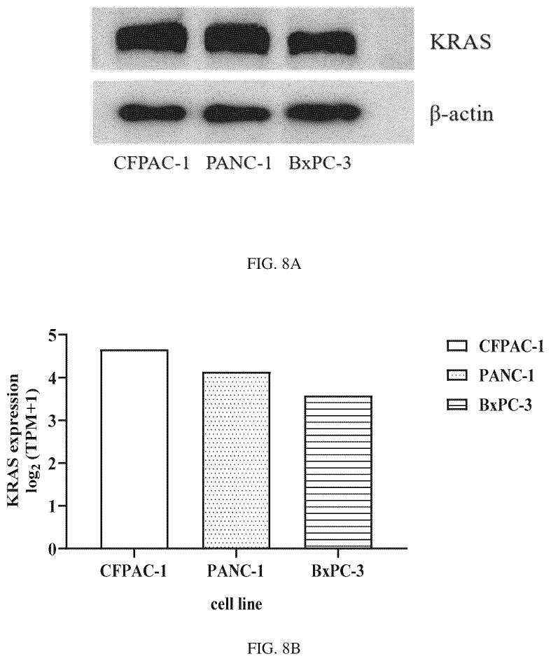

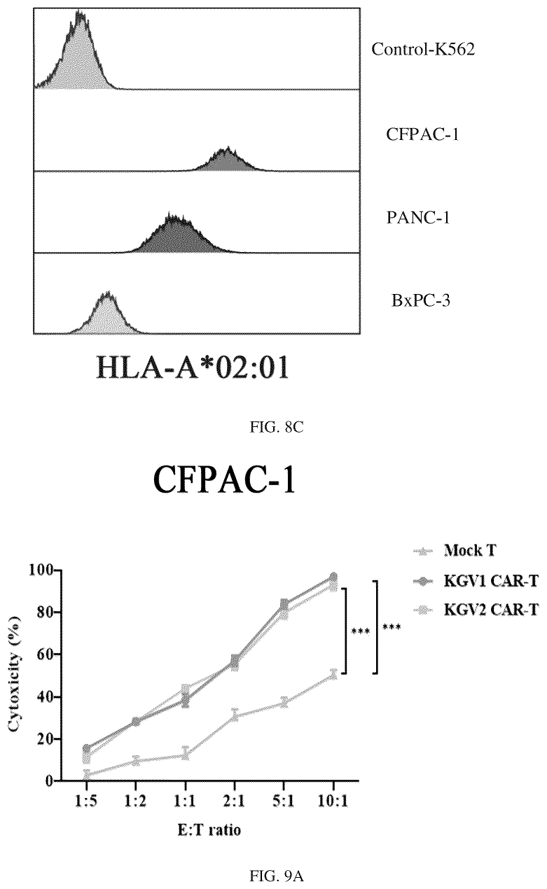

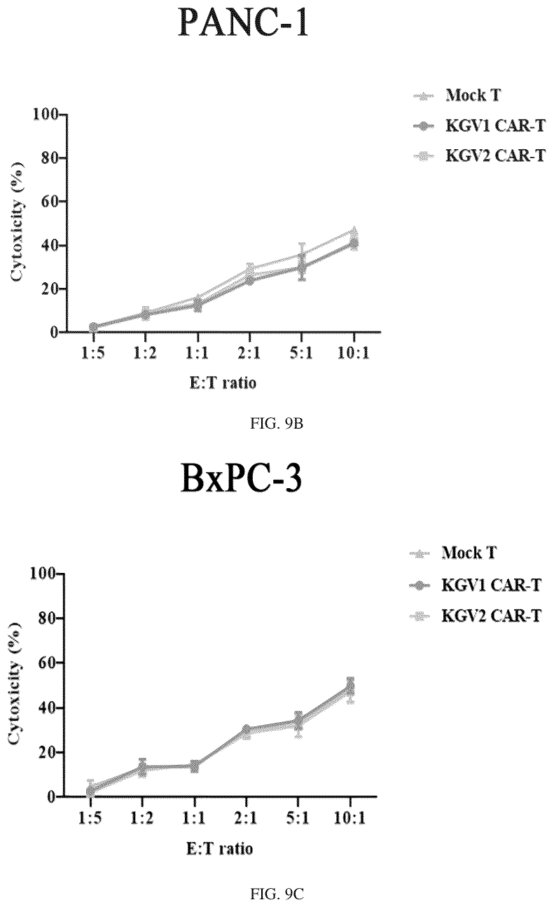

A pancreatic cancer cell line CFPAC-1 was selected as an endogenous target cell. It was verified by sequencing and genomic PCR that the endogenous KRAS G12V mutation and HLA*02:01 both were expressed in the pancreatic cancer cell line CFPAC-1. A pancreatic cancer cell line BxPC-3 was selected as a control target cell. It was verified by sequencing and genomic PCR that the endogenous KRAS G12V mutation and HLA*02:01 were not expressed in the pancreatic cancer cell line BxPC-3. A pancreatic cancer cell line PANC-1 was selected as a control target cell. It was demonstrated by sequencing and genomic PCR that in the pancreatic cancer cell line PANC-1, the endogenous KRAS G12D mutation was expressed, the endogenous KRAS G12V mutation was not expressed, and HLA*02:01 was expressed at a low level. Results are shown in FIG. 9A-FIG. 9C, Table 2, and Table 3. The phenotypic identification of endogenous target cells is shown in FIG. 8A-FIG. 8C. Primer sequences for KRAS sequencing were shown in Table 4.

TABLE 2
KRAS sequencing results of the three pancreatic cancer cell lines

| Cell | KRAS sequence (only | Mutation site |
|---|---|---|
| name | 3 codons before and after G12 were taken) | and type |
| CFPAC-1 | GTTGGAGCTGTTGGCGTAGGC (SEQ ID NO: 21) | G12V |
| PANC-1 | GTTGGAGCTGATGGCGTAGGC (SEQ ID NO: 22) | G12D |
| BxPC-3 | GTTGGAGCTGGTGGCGTAGGC (SEQ ID NO: 23) | WT |

TABLE 3
Sequencing results of HLA-A alleles of
the three pancreatic cancer cell lines

| Cell name | Allele | Allele |
|---|---|---|
| CFPAC-1 | A*02:01:01:01 | A*11:01:01:01 |
| PANC-1 | A*02:01:01:01 | A*03:01:01:01 |
| BxPC-3 | A*01:01:01:01 | A*01:01:01:01 |

TABLE 4
Primer sequences for KRAS sequencing

| Primer name | Primer sequence |
|---|---|
| Forward primer | LEFT: 5′-CCAGGCCTGCTGAAAATGAC-3′ (SEQ ID NO: 24) |
| Reverse primer | RIGHT: 5′-TGGTCCCTCATTGCACTGTA-3′ (SEQ ID NO: 25) |

### Example 10 Analysis of In Vitro Functional Research Results of CAR-T Cells for Tumor

cells exogenously expressing KRAS G12V and HLA-A*02:01 CAR-T cells were co-cultured with K562#4 tumor target cells at effector-to-target (E:T) ratios of 1:2, 1:1, 2:1, 5:1, and 10:1 for 24 h. Then, a killing efficiency was calculated by labeling live target cells with luciferase, and expression levels of cytokines IFN-γ and TNF-α were detected. Blank K562 cells were adopted as a control.

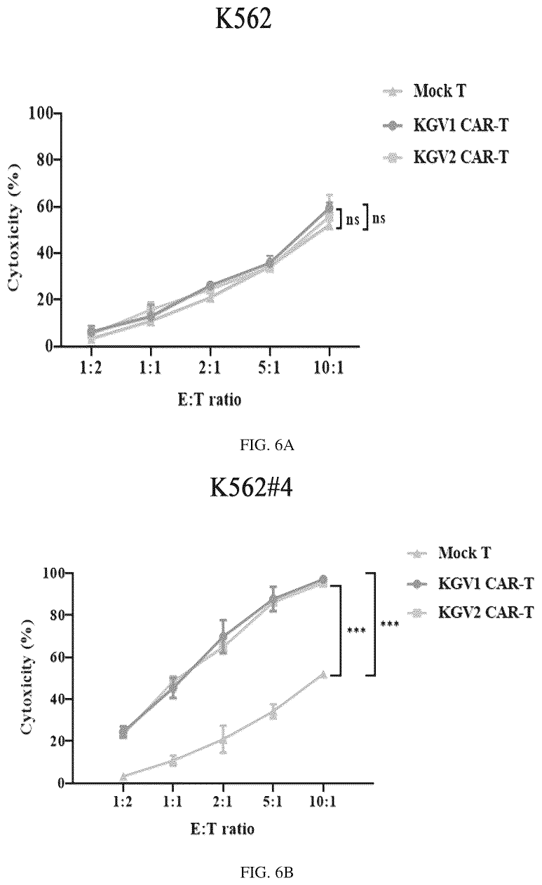

Results showed that killing rates for the two target cells gradually increased with the increase of the effector-to-target ratio. K562 cells were double-negative cells that did not express both KRAS G12V and HLA-A*02:01. The two CAR-T cells did not have a statistical difference from the control group (P>0.05) overall, and exhibited similar killing abilities. The two CAR-T cells both exhibited a significantly-higher killing efficiency for the double-positive cells K562#4 expressing both KRAS G12V and HLA-A*02:01 than for the control group at each effector-to-target ratio, with statistically-significant differences (P<0.05). Moreover, the two CAR-T cells had comparable cytotoxicity. Results are shown in FIG. 6A-FIG. 6B.

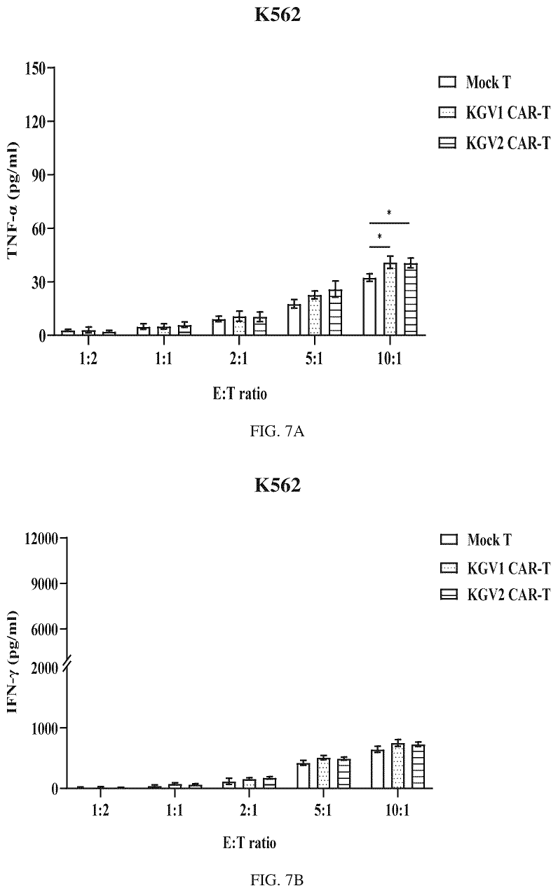

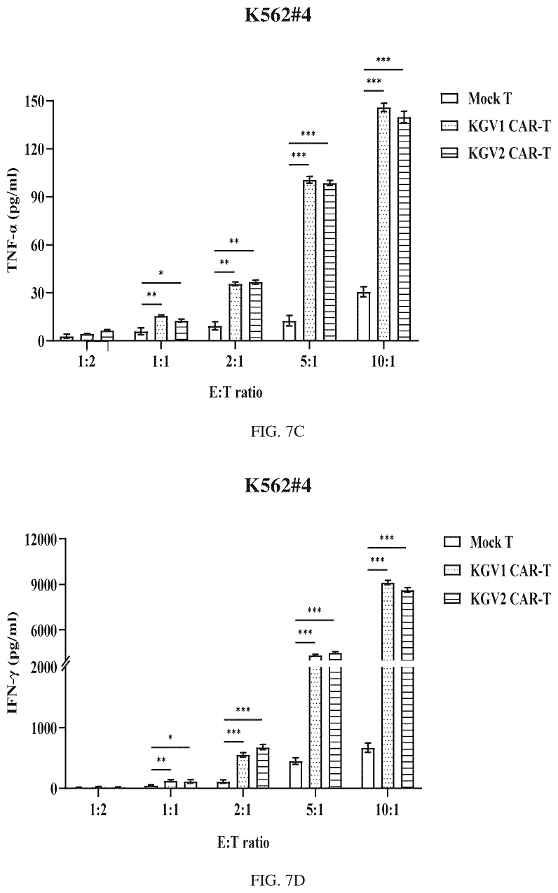

In addition, release levels (concentrations) of cytokines in a supernatant were detected. In double-negative cells K562, although the release levels of cytokines increased with the increase of the effector-to-target ratio, an increase was small. Moreover, there was no significant difference between the three experimental groups. However, in the double-positive cells K562#4, the levels of TNF-α and IFN-γ increased significantly with the increase of the effector-to-target ratio. Moreover, there were significant differences in cytokine concentrations between the two CAR-T cell groups and the control T cell group (P<0.05), indicating that K562#4 cells could activate the two CAR-T cells. It could also be known from the comparison of the two target cell groups that the levels of TNF-α and IFN-γ in the double-positive cells K562#4 were much higher than the levels of TNF-α and IFN-γ in the K562 group, as shown in FIG. 7A-FIG. 7D. According to the in vitro functional experiment, the two CARs designed in the present disclosure both can make T cells specifically recognize an exogenously-expressed target antigen and play a killing role. In addition, the targeted antigen can specifically activate T cells capable of recognizing the targeted antigen and stimulate T cells to release increased cytokines, thereby promoting the rapid expansion of T cells and enhancing the killing effect of T cells.

### Example 11 Analysis of In Vitro Functional Research Results of CAR-T Cells for Tumor

cells endogenously expressing KRAS G12V and HLA-A*02:01 CAR-T cells were co-cultured with each of the three pancreatic cancer cells at effector-to-target ratios of 1:5, 1:2, 1:1, 2:1, 5:1, and 10:1 for 24 h. Then, a killing efficiency was calculated by labeling live target cells with CCK8, and expression levels of cytokines IFN-γ and TNF-α in a supernatant were detected with kits.

The control group reflected an inherent killing ability of T cells for target cells, and killing rates of T cells for the three target cells slowly increased with the increase of the effector-to-target ratio. Killing abilities of KGV1 CAR-T cells and KGV2 CAR-T cells against PANC-1 and BxPC-3 cells also increased slowly with the increase of the effector-to-target ratio, and were similar to a killing ability of the control group. There was no statistical difference between the three groups (P>0.05). That is, KGV1 CAR-T cells and KGV2 CAR-T cells only exerted an inherent killing effect of T cells against PANC-1 and BxPC-3 cells. In the CFPAC-1 cell group, the two CAR-T cells exhibited a significantly-higher killing ability than the control group (P<0.05), and could eliminate almost all target cells at an effector-to-target ratio of 10:1, with a killing rate close to 100%, as shown in FIG. 9A-FIG. 9C. That is, KGV1 CAR-T cells and KGV2 CAR-T cells activated T cells through the specific recognition of a KRAS G12V polypeptide presented by HLA-A*02:01, which imparted a killing potency beyond the inherent killing properties to T cells.

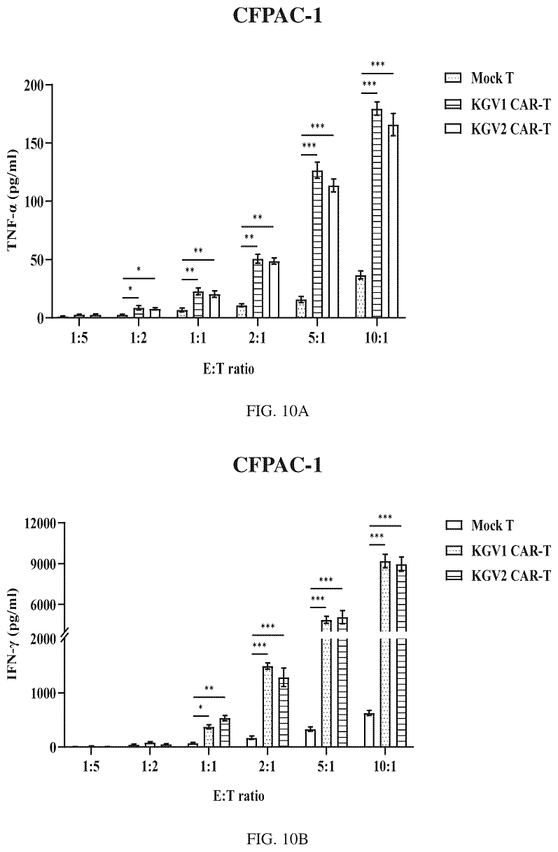

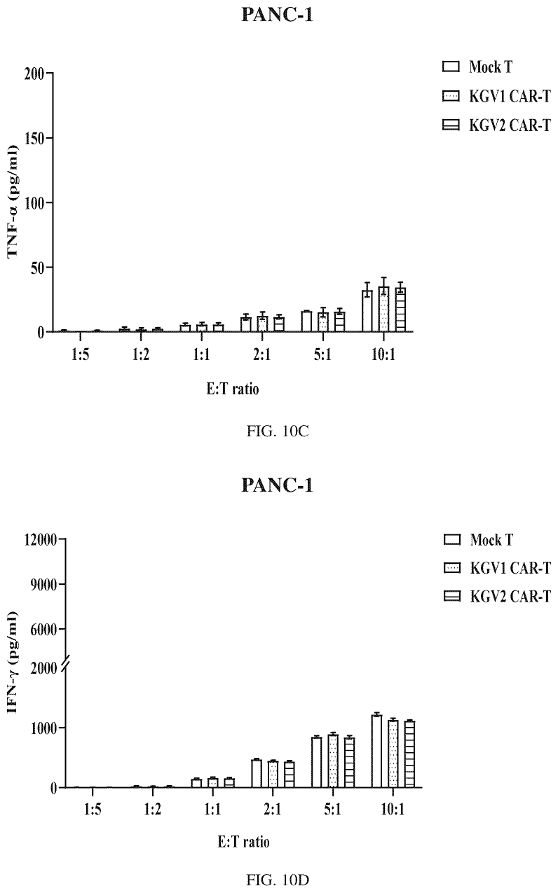

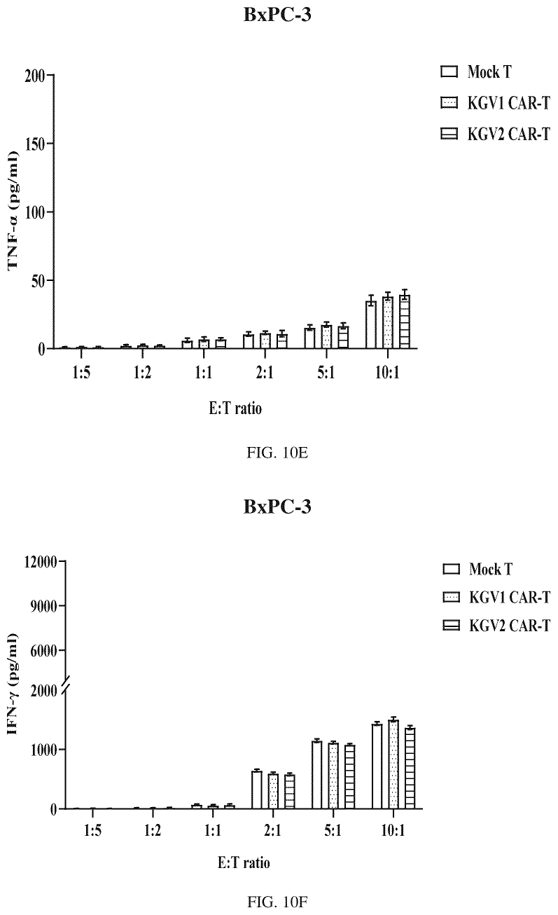

According to the overall cytokine detection results, the level of each cytokine increased with the increase of the effector-to-target ratio. The level of IFN-γ changed with an extremely obvious trend. Levels of IFN-γ released from the two CAR-T cells in the CFPAC-1 cell group were much higher than a level of IFN-γ in the control group, and levels of IFN-γ released from CAR-T cells in the other groups were similar to the level of IFN-γ in the control group. Moreover, release levels of IFN-γ in CAR-T cell groups of CFPAC-1 cells were much higher than release levels of IFN-γ in CAR-T cell groups of the other two pancreatic cancer cells (P<0.05). The results are shown in FIG. 10A-FIG. 10F.

Therefore, both CARs designed and used in the present disclosure can make T cells specifically recognize endogenous target antigens, activate T cells, and exert a killing effect. Since the activation of T cells leads to the production of increased cytokines, the rapid expansion of T cells can be promoted in a feedback manner, and the killing effect of T cells can be enhanced.

The present disclosure has been disclosed above with the preferred examples, but this is not intended to limit the present disclosure. Any technical solution obtained by adopting equivalent replacement or equivalent transformation shall fall within the protection scope of the present disclosure.

1. A single-chain fragment variable targeting KRAS G12V, comprising a heavy chain variable region and a light chain variable region, wherein the heavy chain variable region comprises a VH-CDR1, a VH-CDR2, and a VH-CDR3, and the light chain variable region comprises a VL-CDR1, a VL-CDR2, and a VL-CDR3;
an amino acid sequence of the VH-CDR1 is a sequence set forth in SEQ ID NO: 3, an amino acid sequence of the VH-CDR2 is a sequence set forth in SEQ ID NO: 4, and an amino acid sequence of the VH-CDR3 is a sequence set forth in SEQ ID NO: 5; and
an amino acid sequence of the VL-CDR1 is a sequence set forth in SEQ ID NO: 8, an amino acid sequence of the VL-CDR2 is a sequence set forth in SEQ ID NO: 9, and an amino acid sequence of the VL-CDR3 is a sequence set forth in SEQ ID NO: 10.

2. The single-chain fragment variable targeting KRAS G12V according to claim 1, wherein a full-length amino acid sequence of the heavy chain variable region is an amino acid sequence set forth in SEQ ID NO: 6, or an amino acid sequence set forth in SEQ ID NO: 7;
a full-length amino acid sequence of the light chain variable region is an amino acid sequence set forth in SEQ ID NO: 11, or an amino acid sequence set forth in SEQ ID NO: 12; and
the heavy chain variable region and the light chain variable region are linked by a linker, and the linker comprises three tetrapeptide multimers, and the linker has an amino acid sequence set forth in SEQ ID NO: 13.

3. The single-chain fragment variable targeting KRAS G12V according to claim 2, wherein an amino acid sequence of the single-chain fragment variable is an amino acid sequence set forth in SEQ ID NO: 14 or an amino acid sequence set forth in SEQ ID NO: 15.

4. A chimeric antigen receptor (CAR), comprising an antigen-binding domain, a transmembrane domain, and an intracellular signaling domain, wherein the antigen-binding domain comprises a signal peptide, a Myc tag, and the single-chain fragment variable according to claim 1 for recognizing an antigen.

5. A chimeric antigen receptor-engineered T (CAR-T) cell expressing the CAR according to claim 4.

6. A use of the single-chain fragment variable targeting KRAS G12V according to claim 1 in a preparation of a drug for treating a pancreatic cancer with an endogenous KRAS G12V mutation.

7. A use of the single-chain fragment variable targeting KRAS G12V according to claim 2 in a preparation of a drug for treating a pancreatic cancer with an endogenous KRAS G12V mutation.

8. A use of the single-chain fragment variable targeting KRAS G12V according to claim 3 in a preparation of a drug for treating a pancreatic cancer with an endogenous KRAS G12V mutation.

9. A use of the CAR according to claim 4 in a preparation of a drug for treating a pancreatic cancer with an endogenous KRAS G12V mutation.

10. A use of the CAR-T cell according to claim 5 in a preparation of a drug for treating a pancreatic cancer with an endogenous KRAS G12V mutation.
